SEQUENCE LISTING (10 sequences)

SEQ ID NO: 1 - DNA, 1325 bp, Homo sapiens
  CDS (84)..(1205)
agcatctacaaaggaggaatagtcaaagcagcagcggcggcggcggcggcggcagcagcagcagcagcaggagaccttctctgatggatgacctctgtgaagcaaatggcacttttgccatcagcttatttaaaatattgggggaagaggacaactcaagaaacgtattcttctctcccatgagcatctcctctgccctggccatggtcttcatgggggcaaagggaagcactgcagcccagatgtcccaggcactttgtttatacaaagacggagatattcaccgaggtttccagtcacttctcagtgaagttaacagaactggcactcagtacttgcttagaactgccaacagactctttggagaaaagacgtgtgatttccttccagactttaaagaatactgtcagaagttctatcaggcagagctggaggagttgtcctttgctgaagacactgaagagtgcaggaagcatataaatgactgggtggcagagaagactgaaggtaagatttcagaggtactggatgctgggacagtcgatcccctgacaaagctagtccttgtgaatgccatttatttcaagggaaagtggaatgagcaatttgacagaaagtacacaaggggaatgctctttaaaaccaacgaggaaaaaaagacagtgcagatgatgtttaaggaagctaagtttaaaatggggtatgcggatgaggtacacacccaggtcctggagctgccctatgtggaagaggagctgagcatggtcattctgcttcccgatgacaacacggacctcgccgtggtggaaaaagcacttacatatgagaaattcaaagcctggacaaattcagaaaagttgacaaaaagtaaggttcaagttttccttcccagattaaagctggaggagagttatgacttggagcctttccttcgaagattaggaatgatcgatgcttttgacgaagccaaggcagacttttctggaatgtcaactgagaagaatgtgcctctgtccaaggttgcccacaagtgcttcgtggaggtcaatgaggaaggcacagaggctgccgcagccactgctgtggtcaggaattcccggtgcagcagaatggagccaagattctgtgcagaccacccttttcttttcttcatcaggcgccacaaaaccaactgcatcttgttctgtggcaggttctcttctccgtaaagaggagcaattgctgtacataccctcctttccttctacctatcttgccttaattaacattccctgtgacctagttggtgcagtggcttgaatgccaaaataaagcgtgtgcactgg

SEQ ID NO: 2 - protein, 374 aa, Homo sapiens
MDDLCEANGTFAISLFKILGEEDNSRNVFFSPMSISSALAMVFMGAKGSTAAQMSQALCLYKDGDIHRGFQSLLSEVNRTGTQYLLRTANRLFGEKTCDFLPDFKEYCQKFYQAELEELSFAEDTEECRKHINDWVAEKTEGKISEVLDAGTVDPLTKLVLVNAIYFKGKWNEQFDRKYTRGMLFKTNEEKKTVQMMFKEAKFKMGYADEVHTQVLELPYVEEELSMVILLPDDNTDLAVVEKALTYEKFKAWTNSEKLTKSKVQVFLPRLKLEESYDLEPFLRRLGMIDAFDEAKADFSGMSTEKNVPLSKVAHKCFVEVNEEGTEAAAATAVVRNSRCSRMEPRFCADHPFLFFIRRHKTNCILFCGRFSSP

SEQ ID NO: 3 - DNA, 21 bp, Artificial Sequence
  CAP2 - Exon3 Forward PCR primer
actttcaatttctttgtcatc

SEQ ID NO: 4 - DNA, 22 bp, Artificial Sequence
  CAP2 - Exon3 Reverse PCR Primer
tacaaagcaggagatattcacc

SEQ ID NO: 5 - DNA, 22 bp, Artificial Sequence
  CAP2 - Intron 4 Forward PCR Primer
gaagcatataaatgactgggtg

SEQ ID NO: 6 - DNA, 21 bp, Artificial Sequence
  CAP2 - Intron 4 Reverse PCR Primer
gataagaaatgacagagttgc

SEQ ID NO: 7 - DNA, 18 bp, Artificial Sequence
  CAP2 - Exon 5 Forward PCR Primer
ccaagagaatatttcctg

SEQ ID NO: 8 - DNA, 20 bp, Artificial Sequence
  CAP2 - Exon 5 Reverse PCR Primer
agtcgatcccctgacaaagc

SEQ ID NO: 9 - DNA, 22 bp, Artificial Sequence
  CAP2 - Exon 7 Forward PCR Primer
agctggaggagagttatgactt

SEQ ID NO: 10 - DNA, 22 bp, Artificial Sequence
  CAP2 - Exon 7 Reverse PCR Primer
gcaagataggtagaaggaaagg

(19) United States
(12) Patent Application Publication
(10) Pub. No.: US 2005/0095590 A1
(43) Pub. Date: May 5, 2005

(54) Brain expressed cap-2 gene and protein associated with bipolar disorder

(76) Inventors: Jurgen Peter Lode Del-Favero, Tienen (BE); Christine Van Broeckhoven, Edegem (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/489,185

(22) PCT Filed: Sep. 17, 2002
(86) PCT No.: PCT/EP02/10667

(30) Foreign Application Priority Data
Sep. 12, 2001 (EP) 01203558.0

(51) Int. Cl. C12Q 1/68

(52) U.S. Cl. 435/6

(57) ABSTRACT

We previously identified 18q21.33-q23 as a candidate region for bipolar (BP) disorder and constructed a yeast artificial chromosome (YAC) contig map. In a next step we isolated and analysed all CAG/CTG repeats from this region and excluded them from involvement in BP disorder. Here, in the process of identifying all CCG/CGG repeats from the region, we found a cluster of 6 genes encoding for serpins. For one of them, CAP2 located at 18q21.3, a mutation analysis was performed. Analysis of the thus identified single nucleotide polymorphisms (six in total) revealed a statistically significant association of SNP c.942G>T with BP disorder. It is thus an object of the present invention to provide a method of diagnosing BP or susceptibility to BP in an individual which method comprises determining, in a sample from the individual, the single nucleotide polymorphism in the CAP2 gene of the individual, and determining the status of the individual by reference to polymorphism in the CAP2 gene.

### FIELD OF THE INVENTION

The invention is broadly concerned with the determination of genetic factors associated with psychiatric health. More particularly, the present invention is directed to a human gene which is linked to a mood disorder or related disorder in affected individuals and their families. Specifically, the present invention is directed to a gene encoding cytoplasmic antiproteinase 2 (CAP2). The gene is located on the eighteenth chromosome and is expressed in brain tissue and can be used as a diagnostic marker for bipolar disorder.

### BACKGROUND OF THE INVENTION

Pharmacogenetics Background:

Every individual is a product of the interaction of their genes and the environment. Pharmacogenetics is the study of how genetic differences influence the variability in patients responses to drugs. Through the use of pharmacogenetics, we will soon be able to profile variations between individuals' DNA to predict responses to a particular medicine. Target validation that will predict a well-tolerated and effective medicine for a clinical indication in humans is a widely perceived problem; but the real challenge is target selection. A limited number of molecular target families have been identified, including receptors and enzymes, for which high throughput screening is currently possible. A good target is one against which many compounds can be screened rapidly to identify active molecules (hits). These hits can be developed into optimized molecules (leads), which have the properties of well-tolerated and effective medicines. Selection of targets that can be validated for a disease or clinical symptom is a major problem faced by the pharmaceutical industry. The best-validated targets are those that have already produced well-tolerated and effective medicines in humans (precedent targets). Many targets are chosen on the basis of scientific hypotheses and do not lead to effective medicines because the initial hypotheses are often subsequently disproved.

Two broad strategies are being used to identify genes and express their protein products for use as high-throughput targets. These approaches of genomics and genetics share technologies but represent distinct scientific tactics and investments. Discovery genomics uses the increasing number of databases of DNA sequence information to identify genes and families of genes for tractable or scrollable targets that are not known to be genetically related to disease.

The advantage of information on disease-susceptibility genes derived from patients is that, by definition, these genes are relevant to the patients' genetic contributions to the disease. However, most susceptibility genes will not be tractable targets or amenable to high-throughput screening methods to identify active compounds.

The differential metabolism related to the relevant gene variants can be studied in focused functional genomic and proteomic technologies to discover mechanisms of disease development or progression.

Critical enzymes of receptors associated with the altered metabolism can be used as targets. Gene-to-function-to-target strategies that focus on the role of the specific susceptibility gene variants on appropriate cellular metabolism become important.

Data mining of sequences from the Human Genome Project and similar programmes with powerful bioinformatic tools has made it possible to identify gene families by locating domains that possess similar sequences. Genes identified by these genomic strategies generally require some sort of functional validation or relationship to a disease process. Technologies such as differential gene expression, transgenic animal models, proteomics, in situ hybridization and immunohistochemistry are used to imply relationships between a gene and a disease.

The major distinction between the genomic and genetic approaches is target selection, which genetically defined genes and variant-specific targets already known to be involved in the disease process. The current vogue of discovery genomics for nonspecific, wholesale gene identification, with each gene in search of a relationship to a disease, creates great opportunities for development of medicines.

It is also critical to realize that the core problem for drug development is poor target selection. The screening use of unproven technologies to imply disease-related validation, and the huge investment necessary to progress each selected gene to proof of a concept in humans, is based on an unproven and cavalier use of the word ‘validation’. Each failure is very expensive in lost time and money. For example, differential gene expression (DGE) and proteomics are screening technologies that are widely used for target validation. They detect different levels and/or patterns of gene and protein expression in tissues, which may be used to imply a relationship to a disease affecting that tissue.

Mood Disorder Background:

Mood disorders or related disorders include but are not limited to the following disorders as defined in the Diagnostic and statistical Manual of Mental Disorders, version 4 (DSM-IV) taxonomy DSM-IV codes in parenthesis): mood disorders (296.XX,300.4,311,301.13,295.70), schizophrenia and related disorders (295.XX,297.1,298.8,297.3,298.9), anxiety disorders (300.XX,309.81,308.3), adjustment disorders (309.XX) and personality disorders (codes 301.XX).

The present invention is particularly directed to genetic factors associated with a family of mood disorders known as bipolar (BP) spectrum disorders. Bipolar disorder (BP) is a severe psychiatric condition that is characterized by disturbances in mood, ranging from an extreme state of elation (mania) to a severe state of dysphoria (depression). Two types of bipolar illness have been described: type I BP illness (BPI) is characterized by major depressive episodes alternated with phases of mania, and type II BP illness (BPII), characterized by major depressive episodes alternating with phases of hypomania. Relatives of BP probands have an increased risk for BP, unipolar disorder (patients only experiencing depressive episodes; UP), cyclothymia (minor depression and hypomania episodes; cy) as well as for schizoaffective disorders of the manic (SAm) and depressive (SAd) type. Based on these observations BP, cY, UP and SA are classified as BP spectrum disorders.

The involvement of genetic factors in the etiology of BP spectrum disorders was suggested by family, twin and adoption studies (Tsuang and Faraone (1990), the Genetics of Mood Disorders, Baltimore, The John Hopkins University Press) However, the exact pattern of transmission is unknown. In some studies, complex segregation analysis supports the existence of a single major locus for BP (Spence et al. (1995), Am J.Med. Genet (Neuropsych. Genet.) QQ pp 370-376). Other researchers propose a liability-threshold-model, in which the liability to develop the disorder results from the additive combination of multiple genetic and environmental effects (McGuffin et al. (1994), Affective Disorders; Seminars in Psychiatric Genetics Gaskell, London pp 110-127).

Due to the complex mode of inheritance, parametric and non-parametric linkage strategies are applied in families in which BP disorder appears to be transmitted in a Mendelian fashion. Early linkage findings on chromosomes 11p15 (Egeland et al. (1987), Nature˜pp 783-787) and Xq27-q28 (Mendlewicz'et al. (1987, the Lancet 1 pp 1230-1232; Baron et al. (1987) Nature 12& pp 289-292) have been controversial and could initially not be replicated (Kelsoe et al. (1989) Nature˜pp 238-243; Baron et al. (1993) Nature Genet˜pp 49-55). With the development of a human genetic map saturated with highly polymorphic markers and the continuous development of data analysis techniques, numerous new linkage searches were performed. In several studies, evidence or suggestive evidence for linkage to particular regions on chromosomes 4, 12, 18, 21 and X was found (Black wood et al. (1996) Nature Genetics pp 427-430, Craddock et al. (1994) Brit J. psychiatry˜pp 355-358, Berrettini et al. (1994), Proc Natl Acad Sci USA˜pp 5918-5921, Straub et al. (1994) Nature Genetics pp 291-296 and Pekkarinen et al. (1995) Genome Research 2 pp 105-115). In order to test the validity of the reported linkage results, these findings have to be replicated in other, independent studies.

Recently, linkage of bipolar disorder to the pericentromeric region on chromosome 18 was reported (Berrettini et al. 1994). Also a ring chromosome 18 with break-points and deleted regions at 18pter-p11 and 18q23-qter was reported in three unrelated patients with BP illness or relates syndromes (Craddock et al. 1994). The chromosome 18p linkage was replicated by Stine et al. (1995) Am J. Hum Genet 22 pp 1384-1394, who also reported suggestive evidence for a locus on 18q21.2-q21.32 in the same study.

Interestingly, Stine et al. observed a parent-of-origin effect: the evidence of linkage was the strongest in the paternal pedigrees, in which the proband's father or one of the proband's father's sibs is affected. Several studies described anticipation in families transmitting BP disorder (McInnis et al 1993, Nylander et al 1994) suggesting the involvement of trinucleotide repeat expansions (TREs), considering a number of diseases caused by an expansion of a CAG/CTG, a CCG/CGG or a GAA/TTC repeat show anticipation (reviewed by Margolis et al., 1999). Previous efforts to find potentially expanded repeats have primarily focused on CAG/CTG repeats although the search for CCG/CGG repeats is increasing (Kleiderlein et al 1998, Mangel et al 1998, Eichhammer et al 1998, Kaushik et al 2000). Previously, we reported on a new method for the region specific isolation of triplet repeats: triplet repeat YAC fragmentation (Del Favero et al 1999). This proved to be a valid method for the isolation of CAG/CTG repeats and using this method, we excluded the involvement of CAG/CTG repeats from within 18q21.33-q23 in bipolar disorder (Goossens et al 2000). The present invention adapted the method for the region specific isolation of CCG/CGG repeats and applied it to the chromosome 18q21.33-q23 BP candidate region.

### SUMMARY OF THE INVENTION

The present invention is directed to novel isolated nucleic acid sequence and the cytoplasmic antiproteinase 2 (CAP 2) protein encoded by isolated nucleic acid sequences.

The novel isolated nucleic acid sequence is located at an 8.9 cM chromosome region located between D18S68 and D18S979 at 18q21.33-q23 A physical map was constructed using yeast artificial chromosomes (YACs)(Verheyen et al 1999).

The previously described method was adapted for the region specific isolation of CCG/CGG repeats and applied to the chromosome 18q21.33-q23 BP candidate region. The YAC contig map confirmed the localization within the BP candidate region of a cluster of 6 genes coding for serine proteinase inhibitors (serpins). Serpins are a superfamily of proteolytic proteins with heigh overall homology to α₁₋ proteinase inhibitor. All 6 serpins belong to the ovalbumin family of serpins that lack a typical amino-terminal cleavable signal peptide and can be intracellularly or both. CAP2 or P18 located at 18q21.33, contains a combined CAG-CGG triplet repeat sequence in its 5′UTR region and is expressed in brain. In this study, we determined the genomic organization and exon/intron boundaries of CAP2 and examined the gene by single-strand conformation polymorphism (SSCP) analysis and denaturing high-performance liquid chromatography (DHPLC) for sequence variants. Analysis of six single nucleotide polymorphisms (SNPs) by sequencing, RFLP-PCR or pyrosequencing was performed in a sample of 75 cases and 75 matched controls.

### BRIEF DESCRIPTION OF THE DRAWING

FIG. 1: Minimal YAC tiling path of the 18q21.33-q23 BP candidate region (Verheyen et al 1999). The YACs are represented by solid lines, the CCG/CGG fragmentation products by dotted lines. YAC sizes, between brackets, are estimated by PFGE analysis. Solid circles indicate positive STS/STR hits. Shaded boxes highlight the CCG/CGG repeat and the three CpG islands isolated by YAC fragmentation.

FIG. 2: Genomic structure of Cytoplasmatic antiproteinase 2 (CAP2) gene. Black boxes represent exons and their sizes in bp are indicated above the box. Introns sizes are in kb. The combined CAG-CGG repeat is indicated. Transcription initiation and stop codons are indicated.

### DETAILED DESCRIPTION OF THE INVENTION

The present invention is directed to a novel isolated nucleic acid sequence comprising gene located at the 18q chromosomal candidate region of chromosome 18.

The gene is located at a chromosomal region associated with mood disorders such as bipolar spectrum disorders and therefore is useful as a diagnostic marker for bipolar spectrum disorders. The region in question when removed from the totality of the human genome may also be used to locate, isolate and sequence other genes which influences psychiatric health and mood.

Specifically the BP candidate region contains the gene coding for cytoplasmic antiproteinase 2 (CAP2), a brain expressed serpin implicated in a number of intra-and extracellular functions. In this study we determined the genomic organization of CAP2 and defined all intron/exon boundaries. CAP2 comprises 7 exons within an estimated 17-kb genomic region.

Mutation analysis of CAP2 identified 3 non-synonymous single nucleotide polymorphism (SNPs): c.203G>A (Arg69Gln), c.910A>G (Thr304Ala) and c.1076G>A (Arg359His); 2 synonymous SNPs c. 477>G and c.942>T and 1 intronic SNP IVS4+98A>G. Analysis of CAP2 polymorphisms in unrelated BP cases and matched controls showed a statistical significant difference with SNP c.942C>T in allele aand genotype frequencies (p=0.03).

Isolation and Identification of Novel Gene:

Standard procedures well-known to one skilled in the art were applied to the identified YAC clones and, where applicable, to the DNA from an individual afflicted with a mood disorder as defined herein, in the process of identifying and characterizing the relevant gene. For example, the inventors are able to make use of the previously identified apparent association between trinucleotide repeat expansions (TRE) within the human genome and the phenomenon of anticipation in mood disorders (Lindblad et al. (1995), Neurobiology of Disease 2. pp 55-62 and O'Donovan et al. (1995), Nature Genetics IQ pp 380-381) to screen for TRE's in the selected YAC clones in order to identify candidate genes in the region of interest on human chromosomel 8. A variety of other known procedures can also be applied to the said YAC clones to identify the candidate gene as discussed below.

Accordingly, in a first aspect the present invention comprises the use of an 8.9 cM region of human chromosome 18q disposed between polymorphic markers D18S68 and D18S979 or a fragment thereof for identifying at least one human gene, including mutated and polymorphic variants thereof, which is associated with mood disorders or related disorders as defined above. As will be described below, the present inventors have identified this candidate region of chromosome 18q for such a gene, by analysis of co-segregation of bipolar disease in family MAD31 with 12 STR polymorphic markers previously located between D18S51 and D18S61 and subsequent allele sharing analysis.

Particular YACs covering the candidate region which may be used in accordance with the present invention are 961.h-9, 942-c.3, 766-f-12, 731-c-7, 907.e.1, 752-g-8 and 717-d-3, preferred ones being 961h-9, 766.f.12 and 907-e.1 since these have the minimum tiling path across the candidate region. suitable YAC clones for use are those having an artificial chromosome spanning the refined candidate region between D18S68 and D18S979.

There are a number of methods which can be applied to the candidate regions of chromosome 18q as defined above, whether or not present in a YAC, to identify a candidate gene or genes associated with mood disorders or related disorders. For example, as aforesaid, there is an apparent association between the extent of trinucleotide repeat expansions (TRE) in the human genome and the presence of mood disorders.

Accordingly, in a third aspect the present invention comprises a method of identifying at least one human gene, including mutated and polymorphic variants thereof, which is associated with a mood disorder or related disorder as defined herein which comprises detecting nucleotide triplet repeats in the region of human chromosome 18q disposed between polymorphic markers D18S68 and D 18S979.

An alternative method of identifying said gene or genes comprises fragmenting a YAC clone comprising a portion of human chromosome 18q disposed between polymorphic markers D18S60 and D18S61, for example one or more of the seven aforementioned YAC clones, and detecting any nucleotide triplet repeats in said fragments, in particular repeats of CAG or CTG. Nucleic acid probes comprising at least 5 and preferably at least 10 CTG and/or CAG triplet repeats are a suitable means of detection when appropriately labelled. Trinucleotide repeats may also be determined using the known RED (repeat expansion detection) system (Shalling et al. (1993), Nature Genetics˜pp 135-139).

In a fourth embodiment the invention comprises a method of identifying at least one gene, including mutated and polymorphic variants thereof, which is associated with a mood disorder or related disorder and which is present in a YAC clone spanning the region of human chromosome 18q between polymorphic markers D18S60 and D18S61, the method comprising the step of detecting the expression product of a gene incorporating nucleotide triplet repeats by use of an antibody capable of recognizing a protein with anamino acid sequence comprising a string of at least 8, but preferably at least 12, continuous glutamine residues. Such a method may be implemented by sub-cloning YAC DNA, for example from the seven aforementioned YAC clones, into a human DNA expression library. A preferred means of detecting the relevant expression product is by use of a monoclonal antibody, in particular mAB1C2, the preparation and properties of which are described in International Patent. Application Publication No WO 97/17445.

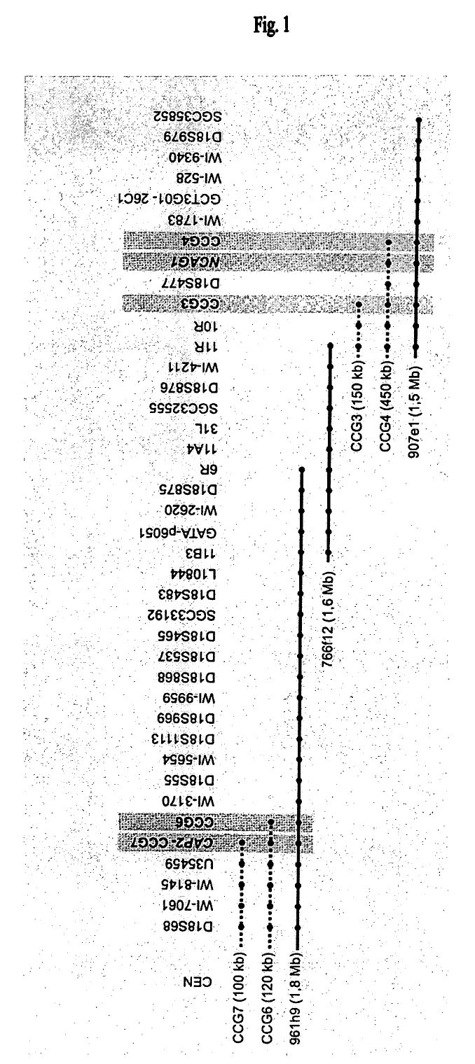

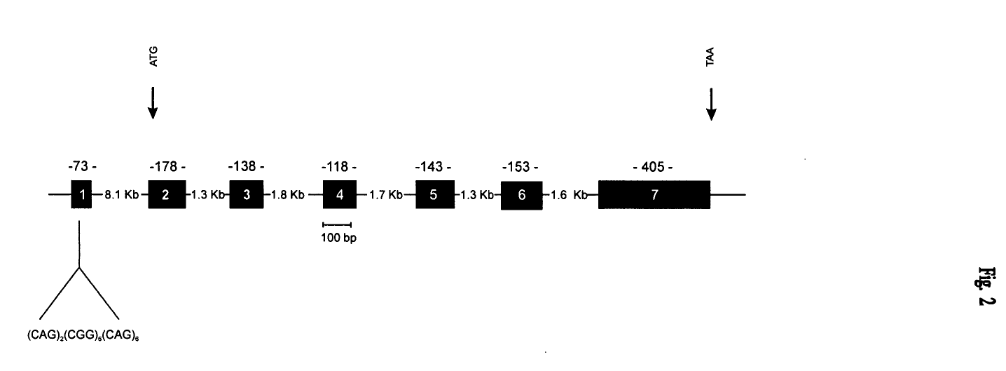

Further embodiments of the present invention relate to methods of identifying the relevant gene or genes which involve the (sub-)cloning of (YAC) DNA as defined above into vectors such as BAC (bacterial artificial chromosome) or PAC (P1 or phage artificial chromosome) or cosmid vectors such as exon-trap cosmid vectors. The starting point for such methods is the construction of a contig map of the region of human chromosome 18q between polymorphic markers D18S60 and D18S61. To this end the present inventors have sequenced the end regions of the fragment of human DNA in each of the seven aforementioned YAC clones and these sequences are disclosed herein. Following sub-cloning of YAC DNA into other vectors as described above, probes comprising these end sequences or portions thereof, in particular those sequences shown in FIGS. 1 to 11 herein, together with any known sequenced tagged site (STS) in this region, as described in the YAC clone contig shown herein, as can be used to detect overlaps between said sub-clones and a contig map can be constructed. Also the known sequences in the current YAC contig can be used for the generation of contig map sub-clones.

One route by which a gene or genes which is associated with a mood disorder or associated disorder can be identified is by use of the known technique of exon trapping. This is an artificial RNA splicing assay, most often making use in current protocols of a specialized exon-trap cosmid vector. The vector contains an artificial mini-gene consisting of a segment of the SV40 genome containing an origin of replication and a powerful promoter sequence, two splicing-competent exons separated by an intron which contains a multiple cloning site and an SV40 polyadenylation site.

The YAC DNA is sub-cloned in the exon-trap vector and the recombinant DNA is transfected into a strain of mammalian cells. Transcription from the SV40 promoter results in an RNA transcript which normally splices to include the two exons of the minigene. If the cloned DNA itself contains a functional exon, it can be spliced to the exons present in the vector's minigene. Using reverse transcriptase a cDNA copy can be made and using specific PCR primers, splicing events involving exons of the insert DNA can be identified. Such a procedure can identify coding regions in the YAC DNA which can be compared to the equivalent regions of DNA from a person afflicted with a mood disorder or related disorder to identify the relevant gene.

Accordingly, in a fifth aspect the invention comprises a method of identifying at least one human gene, including mutated variants and polymorphisms thereof, which is associated with a mood disorder or related disorder which comprises the steps of:
(1) transfecting mammalian cells with exon trap cosmid vectors prepared and mapped as described above;
(2) culturing said mammalian cells in an appropriate medium;
(3) isolating RNA transcripts expressed from the SV40 promoter;
(4) preparing cDNA from said RNA transcripts;
(5) identifying splicing events involving exons of the DNA sub-cloned into said exon trap cosmid vectors to elucidate positions of coding regions in said sub-cloned DNA;
(6) detecting differences between said coding regions and equivalent regions in the DNA of an individual afflicted with said mood disorder or related disorder; and
(7) identifying said gene or mutated or polymorphic variant thereof which is associated with said mood disorder or related disorders.

As an alternative to exon trapping the YAC DNA may be sub-cloned into BAC, PAC, cosmid or other vectors and a contig map constructed as described above. There are a variety of known methods available by which the position of relevant genes on the sub-cloned DNA can be established as follows:
(a) cDNA selection or capture (also called direct selection and cDNA selection): this method involves the forming of genomic DNA/cDNA heteroduplexes by hybridizing a cloned DNA (e.g. an insert of a YAC DNA), to a complex mixture of cDNAs, such as the inserts of all cDNA clones from a specific (e.g. brain) cDNA library. Related sequences will hybridize and can be enriched in subsequent steps using biotin-streptavidine capturing and PCR (or related techniques);
(b) hybridization to mRNA/cDNA: a genomic clone (e.g. the insert of a specific cosmid) can be hybridized to a Northern blot of mRNA from a panel of culture cell lines or against appropriate (e.g. brain) cDNA libraries. A positive signal can indicate the presence of a gene within the cloned fragment;
(c) CpG island identification: CpG or HTF islands are short (about 1 kb) hypomethylated GC-rich (>60%) sequences which are often found at the 5′ ends of genes. CpG islands often have restriction sites for several rare-cutter restriction enzymes. Clustering of rare-cutter restriction sites is indicative of a CpG island and therefore of a possible gene. CpG islands can be detected by hybridization of a DNA clone to Southern blots of genomic DNA digested with rare-cutting enzymes, or by island-rescue PCR (isolation of CpG islands from YACs by amplifying sequences between islands and neighbouring Alu-repeats);
(d) zoo-blotting: hybridizing a DNA clone (e.g. the insert of a specific cosmid) at reduced stringency against a Southern blot of genomic DNA samples from a variety of animal species. Detection of hybridization signals can suggest conserved sequences, indicating a possible gene. Accordingly, in a sixth aspect the invention comprises a method of identifying at least one human gene including mutated and polymorphic variants thereof which is associated with a mood disorder or related disorder which comprises the steps of:
(1) sub-cloning the YAC DNA as described above into a cosmid, BAC, PAC or other vector;
(2) using the nucleotide sequences or any other sequenced tagged site (STS) in this region as in the YAC clone contig described herein, or part thereof consisting of not less than 14 contiguous bases or the complement thereof, to detect overlaps amongst the sub-clones and construct a map thereof;
(3) identifying the position of genes within the sub-cloned DNA by one or more of CpG island identification, zoo-blotting, hybridization of the sub-cloned DNA to a cDNA library or a Northern blot of mRNA from a panel of culture cell lines;
(4) detecting differences between said genes and equivalent region of the DNA of an individual afflicted with a mood disorder or related disorder; and
(5) identifying said gene which is associated with said mood disorders or related disorders.

If the cloned YAC DNA is sequenced, computer analysis can be used to establish the presence of relevant genes. Techniques such as homology searching and exon prediction may be applied.

Once a candidate gene has been isolated in accordance with the methods of the invention more detailed comparisons may be made between the gene from a normal individual and one afflicted with a mood disorder such as a bipolar spectrum disorder. For example, there are two methods, described as “mutation testing”, by which a mutation or polymorphism in a DNA sequence can be identified. In the first the DNA sample may be tested for the presence or absence of one specific mutation but this requires knowledge of what the mutation might be. In the second a sample of DNA is screened for any deviation from a standard (normal) DNA. This latter method is more useful for identifying candidate genes where a mutation is not identified in advance. In addition the following techniques may be further applied to a gene identified by the above-described methods to identify differences between genes from normal or healthy individuals and those afflicted with a mood disorder or related disorder:
(a) Southern blotting techniques: a clone is hybridized to nylon membranes containing genomic DNA digested with different restriction enzymes of patients and healthy individuals. Large differences between patients and healthy individuals can be visualized using a radioactive labelling protocol;
(b) heteroduplex mobility in polyacrylamide gels: this technique is based on the fact that the mobility of heteroduplexes in non-denaturing polyacrylamide gels is less than the mobility of homoduplexes. It is most effective for fragments under 200 bp;
(c) single-strand conformational polymorphism analysis (SSCP or SSCA): single stranded DNA folds up to form complex structures that are stabilized by weak intramolecular bonds.

The electrophoretic mobilities of these structures on non-denaturing polyacrylamide gels depends on their chain lengths and on their conformation;
(d) chemical cleavage of mismatches (CCM): a radiolabelled probe is hybridized to the test DNA, and mismatches detected by a series of chemical reactions that cleave one strand of the DNA at the site of the mismatch. This is a very sensitive method and can be applied to kilobase-length samples;
(e) enzymatic cleavage of mismatches: the assay is similar to CCM, but the cleavage is performed by certain bacteriophage or eukaryotic enzymes.
(f) denaturing gradient gel electrophoresis: in this technique, DNA duplexes are forced to migrate through an electrophoretic gel in which there is a gradient of increasing amounts of a denaturant (chemical or temperature). Migration continues until the DNA duplexes reach a position on the gel wherein the strands melt and separate, after which the denatured DNA does not migrate much further. A single base pair difference between a normal and a mutant DNA duplex is sufficient to cause them to migrate to different positions in the gel;
(g) direct DNA sequencing.

A more detailed discussion of these suitable assay techniques is provided below.

GENOTYPING As used herein, the term “genotyping” means determining whether a CAP2 encoding polynucleotide includes a thymidine (T) at position 942. The term “genotyping” is synonymous with terms such as “genetic testing”, “genetic screening”, “determining or identifying an allele or polymorphism”, “molecular diagnostics” or any other similar phrase.

Any method capable of distinguishing nucleotide differences in the appropriate sample DNA sequences may also be used. In fact, a number of known different methods are suitable for use in genotyping (that is, determining the genotype) for a CAP2 encoding polynucleotide of the present invention. These methods include but are not limited to direct sequencing, PCR-RFLP, ARMS-PCR, Taqman™, Molecular beacons, hybridization to oligonucleotides on DNA chips and arrays, single nucleotide primer extension and oligo ligation assays.

GENOTYPE SCREENING In one embodiment, the present invention provides a method for genotype screening of a nucleic acid comprising a CAP2 encoding polynucleotide from an individual. The methods for genotype screening of a nucleic acid comprising a CAP2 encoding polynucleotide from an individual may require amplification of a nucleic acids from a target sample from that individual.

TARGET SAMPLE The target samples of the present invention may be any target nucleic acid comprising a CAP2 encoding polynucleotide from an individual being analyzed. For assay of such nucleic acids, virtually any biological sample (other than pure red blood cells) is suitable. For example, convenient target samples include but are not limited to whole blood, leukocytes, semen, saliva, tears, urine, faecal material, sweat, buccal, skin and hair. For assay of cDNA or mRNA, the target sample is typically obtained from a cell or organ in which the target nucleic acid is expressed.

GENOTYPING SNPS A number of different methods are suitable for use in determining the genotype for an SNP. These methods include but are not limited to direct sequencing, PCR-RFLP, ARMS-PCR, Taqman™, Molecular beacons, hybridization to oligonucleotides on DNA chips and arrays, single nucleotide primer extension and oligo ligation assays. Any method capable of distinguishing single nucleotide differences in the appropriate DNA sequences may also be used.

AMPLIFICATION As used herein, the term “amplification means nucleic acid replication involving template specificity. The template specificity relates to a “target sample” or “target sequence” specificity. The target sequences are “targets” in the sense that they are sought to be sorted out from other nucleic acids. Consequently, amplification techniques have been designed primarily for sorting this out. Examples of amplification methods include but are not limited to polymerase chain reaction (PCR), polymerase chain reaction of specific alleles (PASA), ligase chain reaction (LCR), transcription amplification, self-sustained sequence replication and nucleic acid based sequence amplification (NASBA).

TAQMAN Suitable means for determining genotype may be based on the Taqman™ technique. The Taqman™ technique is disclosed in the following U.S. Pat. Nos. 4,683,202; 4,683,195 and 4,965,188. The use of uracil N-glycosylase which is included in Taqman™ allelic discrimination assays is disclosed in U.S. Pat. No. 5,035,996.

PCR PCR techniques are well known in the art (see for example, EP-A-0200362 and EP-A-0201184 and U.S. Pat. Nos. 4,683,195 and 4,683,202). The process for amplifying the target sequence consists of introducing an excess of two oligonucleotide primers to the DNA mixture containing the desired target sequence, followed by a precise sequence of thermal cycling in the presence of a DNA polymerase. With PCR, it is possible to amplify a single copy of a specific target sequence in, for example, genomic DNA to a level detectable by several different methodologies (such as hybridisation with a labelled probe, incorporation of biotinylated primers followed by avidin-enzyme conjugate detection and incorporation of 32P labelled deoxynucleotide triphosphates, such as dCTP or dATP, into the amplified sequence). Alternatively, it is possible to amplify different polymorphic sites (markers) with primers that are differentially labelled and thus can each be detected. One means of analysing multiple markers involves labelling each marker with a different fluorescent probe. The PCR products are then analysed on a fluorescence based automated sequencer. In addition to genomic DNA, any oligonucleotide sequence may be amplified with the appropriate set of primer molecules. In particular, the amplified segments created by the PCR process itself are, themselves, efficient templates for subsequent PCR amplifications. By way of example, PCR can also be used to identify primers for amplifying suitable sections of a CAP2 encoding polynucleotide in or from a human.

PRIMERS The present invention also provides a series of useful primers.

As used herein, the term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template.

The term “primer site” refers to the area of the target DNA to which a primer hybridizes.

The term “primer pair” means a set of primers including a 5′ upstream primer that hybridizes with the 5′ end of the DNA sequence to be amplified and a 3′ downstream primer that hybridizes with the complement of the 3′ end of the sequence to be amplified.

The primers of the present invention may be DNA or RNA, and single-or double-stranded. Alternatively, the primers may be naturally occurring or synthetic, but are typically prepared by synthetic means.

PRIMER HYBRIDISATION CONDITIONS As used herein, the term “hybridisation” refers to the pairing of complementary nucleic acids. Hybridisation and the strength of hybridisation (i.e. the strength of association between the nucleic acids) is impacted by such factors as the degree of complementarity between nucleic acids, stringency of conditions involved, the melting temperature (Tm) of the formed hybrid and the G:C ratio within the nucleic acids.

As used herein, the term “stringency” is used in reference to the conditions of temperature, ionic strength and the presence of other compounds such as organic solvents under which the nucleic acid hybridizations are conducted.

Hybridizations are typically performed under stringent conditions, for example, at a salt concentration of no more than IM and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific primer hybridizations.

ALLELE SPECIFIC PRIMERS An allele-specific primer hybridises to a site on target DNA overlapping a polymorphism and only primes amplification of an allelic form to which the primer exhibits perfect complementarity (See Gibbs, Nucleic Acid Res. 17, 2427-2448 (1989)). This primer may be used in conjunction with a second primer which hybridises at a distal site. Amplification proceeds from the two primers leading to a detectable product signifying the particular allelic form is present. A control may be performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′—most position of the oligonucleotide aligned with the polymorphism because this position is most destabilizing to elongation from the primer (see, for example WO 93/22456).

Hybridisation probes capable of specific hybridisation to detect a single base mismatch may be designed according to methods known in the art and described in Maniatas et al Molecular Cloning: A Laboratory Manual, 2nd Ed (1989) Cold Spring Harbour.

(i) PCR PRIMERS Preferably the screening is carried out using PCR primers designed to amplify portions of the human a CAP2 encoding polynucleotide (gene) that include nucleotide 942.

Examples of such PCR primers are shown as SEQ ID's Nos. 9 and 10.

DETECTION OF POLYMORPHISMS IN AMPLIFIED TARGET SEQUENCES The amplified nucleic acid sequences may be detected using procedures including but not limited to allele-specific probes, tiling arrays, direct sequencing, denaturing gradient gel electrophoresis and single-strand conformation polymorphism (SCCP) analysis.

ALLELE-SPECIFIC PROBES Allele-specific probes can be designed that hybridize to a segment of target DNA from one individual but do not hybridize to the corresponding segment from another individual due to the presence of different polymorphic forms in the respective segments from the two individuals.

As used herein, the term “probe” refers to an oligonucleotide (i.e. a sequence of nucleotides), whether occurring naturally as in a purified restriction digest or produced synthetically, which is capable of hybridizing to another oligonucleotide sequence of interest. Probes are useful in the detection, identification and isolation of particular gene sequences. The hybridisation probes of the present invention are typically oligonucleotides capable of binding in a base-specific manner to a complementary strand of nucleic acid.

The probes of the present invention may be labeled with any “reporter molecule” so that it is detectable in any detection system, including but not limited to enzyme (for example, ELISA, as well as enzyme based histochemical assays), fluorescent, radioactive and luminescent systems. The target sequence of interest (that is, the sequence to be detected) may also be labeled with a reporter molecule. The present invention is not limited to any particular detection system or label.

The hybridization conditions chosen for the probes of the present invention are sufficiently stringent that there is a significant difference in hybridization intensity between alleles, and preferably an essentially binary response, whereby a probe hybridizes to only one of the alleles. The typical hybridization conditions are stringent conditions as set out above for the allele specific primers of the present invention so that a one base pair mismatch may be determined.

TILING ARRAYS The polymorphisms of the present invention may also be identified by hybridisation to nucleic acid arrays, some example of which are described in WO 95/11995. The term “tiling” generally means the synthesis of a defined set of oligonucleotide probes that is made up of a sequence complementary to the sequence to be analysed (the “target sequence”), as well as preselected variations of that sequence. The variations usually include substitution at one or more base positions with one or more nucleotides.

DIRECT SEQUENCING The direct analysis of the sequence of polymorphisms of the present invention may be accomplished using either the dideoxy chain termination method or the Maxam Gilbert method (see Sambrook et al., Molecular Cloning, A Laboratory Manual (2nd Ed., CSHP, New York 1989) or using, for example, Standard ABI sequencing technology using Big Dye Terminator cycle sequencing chemistry analyzed on an ABI Prism 377 DNA sequencer. Preferably, the polymorphism used in the assays of the present invention are identified by the presence or absence of the fragments generated by PstI restriction analysis of the identified sequences.

1.5 DENATURING GRADIENT GEL ELECTROPHORESIS Amplification products of the present invention, which are generated using PCR, may also be analyzed by the use of denaturing gradient gel electrophoresis. Different alleles may be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

SINGLE-STRAND CONFORMATION POLYMORPHISM (SCCP) ANALYSIS Alleles of target sequences of the present invention may also be differentiated using single-strand conformation polymorphism (SCCP) analysis, which identifies base differences by alteration in electrophoretic migration of single stranded PCR products, as described in Orita et al., Proc. Nat. Acad. Sci. 86, 2766-2770(1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products may be related to base-sequence difference between alleles of target sequences.

IDENTIFYING DIFFERENCES BETWEEN TEST AND CONTROL SEQUENCES These detection procedures for amplified nucleic acid sequences may be used to identify difference of one or more points of variation between a reference and test nucleic acid sequence or to compare different polymorphic forms of the CAP2 gene from two or more individuals.

REFERENCE NUCLEIC ACID SEQUENCES As used herein the term “reference nucleic acid sequence” means a control nucleic acid sequence such as a control DNA sequence representing one or more individuals homozygous for each of the alleles being tested in that assay. By way of example, control DNA sequences may include but are not limited to: (i) a genomic DNA from homozygous individuals; (ii) a PCR product containing a relevant SNP amplified from homozygous individuals; or (iii) a DNA sequence containing a relevant SNP that has been cloned into a plasmid or other suitable vector. The control sample may also be an alleleic ladder comprising a plurality of alleles from known set of alleles. There may be a plurality of control samples, each containing different alleles or sets of alleles. Other reference/control samples typically include diagrammatic representations, written representations, templates or any other means suitable for identifying the presence of a polymorphism in a PCR product or other fragment of nucleic acid. The terms “reference nucleic acid sequence”, reference samples and control samples are used interchangeable throughout the text.

H. THERAPEUTIC USES An aspect of the invention provides screening an individual for a predisposition to bipolar mood disorder and, if a polynucleotidetic predisposition is identified, treating that individual to delay or reduce or prevent the bipolar mood disorder.

In an embodiment of this aspect of the invention, the predisposition of an individual to bipolar mood disorder is assessed by determining whether that individual is homozygous for a CAP2 encoding polynucleotide in which nucleotide 942 is thymidine (T), is heterozygous for a CAP2 encoding polynucleotide in which guanosine (G) at position 942 is replaced by thymidine (T), or is homozygous for a CAP2 encoding polynucleotide in which nucleotide 942 is guanosine (G) using methods of detection discussed above.

Thus, an individual who is T/T homozygous at position 942, for the polymorphism is classified as being at highest risk. An individual being G/T heterozygous is classified as having moderate risk. An individual being G/G homozygous is classified as being in the lowest risk category.

Optionally, the assessment of an individual's risk factor is calculated by reference both to the presence of a CAP2 encoding polynucleotide polymorphism and also to other known polynucleotidetic or physiological or other indications. The invention in this way provides further information on which measurement of an individual's risk can be based.

General methodology reference Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc.

It will be appreciated that with respect to the methods described herein, in the step of detecting differences between coding regions from the YAC and the DNA of an individual afflicted with a mood disorder or related disorder, the said individual may be anybody with the disorder and not necessary a member of family MAD31.

In accordance with further aspects the present invention provides an isolated human gene and variants thereof associated with a mood disorder or related disorder and which is obtainable by any of the above described methods, an isolated human protein encoded by said gene and a cDNA encoding said protein.

Once a gene has been identified a number of methods are available to determine the function of the encoded protein. These methods are described by Eisenberg et al (Nature vol. 15, June 2000) and is herein incorporated by reference. One method involves a computational method that reveals functional linkages from genome sequences and is called the gene neighbor method. If in several genomes the genes that encode two proteins are neighbors on the chromosome, the proteins tend to be functionally linked. This method can be powerful in uncovering functional linkages in prokaryotes, where operons are common, but also shows promise for analysing interacting proteins in eukaryotes.

CAP-2 Gene

The complete intron-exon structure of the Cytoplasmic antiproteinase 2 gene (CAP2), which contains a 5′UTR and 6 coding exons spanning a genomic of 17.1 kb is herein disclosed. To size the introns, different combinations of primers spanning the CAP2 cDNA sequence are used. In this way, the size and exon-intron boundary sequences of 5 introns were derived. The 5′ donor and 3′ acceptor sites at the splice junctions correlated with consensus sequences (Table 1). The first 5′UTR exon is very small (73 bp) and contains a (CAG)₂(CGG)₆(CAG)₆ sequence which proved to be polymorphic but not expanded in the MAD 31 Belgian family nor in the affected and the control population.

The CAP2 derived amino acid sequence exhibits a high degree of identity to other human members of the ovalbumin family of cytoplasmic serpins including Placental thrombin inhibitor or P16 (68% identity) and proteinase inhibitor 9 P19 (63%). 5 exonic polymorphisms were identified from which 3 result in aminoacid change. Alignment of the deduced primary structure of CAP2 with the amino acid sequences of P16 and P19, showed that, at amino acid position 68, CAP2 can either be Arg or Gln and in P16 is Gln. Similarly, at amino acid position 359, CAP2 exhibits either Arg or His and PI6 exhibits His.¹⁵ In addition, at amino acid position 304, CAP2 exhibits either Thr or Ala and P19 exhibits Ala. By contrary, at amino acid position 314, CAP2 exhibits Ala while P16 and P19 exhibit Val.

Two of these variants (c.910A>G and c.942C>T) detected by DHPLC were not previously identified by SSCP analysis.

An association analysis in a 75/75 case-control sample of Belgian origin was applied. Cases and controls were strictly matched for ethnicity, gender and age. Comparison of the allele and genotype frequencies of the 6 SNPs indicated no significant association between patients and controls for 5 of the 6 SNPs. The frequency of the SNP c.942C>T substitution in exon 7 was significantly different between BP patients and controls: BP patients had a higher frequency of the T allele when compared to controls (p=0.03). Although the 6 SNPs are located within the same gene, 4 of them were not in linkage disequilibrium. There was a very strong LD between the SNP c.203G>A and the SNP IVS4+98A>G, but only in controls. In BP cases, the LD between these 2 SNPs was weak. In addition, no LD was found between the CAP2-CAG-CGG repeat and the CAP2 SNPs. The fact that no strong evidence for association was found with BP disorder and the CAP2 SNPs, together with the lack of significant LD results in our population, suggests that the CAP2 might not play a major role in the etiology of BP disorders.

In one affected individual of family MAD31, a proximal recombination occurred between D18S68 and D18S969.¹¹ CAP2 gene is located between these two markers. The CAP2-SNPs indicated that this proximal recombination occurred downstream of the gene.

### EXAMPLE 1

A. Family, Patients and Control Subjects

The pedigree and the clinical diagnoses in MAD 31, a Belgian family with a BPII proband, were described in detail elsewhere.¹⁰ Briefly, the different clinical diagnoses in family MAD31 are as follows: 1 BPI, 2 BPII, 2 UP, 4 major depressive disorder (MDD), 1 schizoaffective maniac (SAm) and 1 schizoaffective depressive (SAd).

The case-control sample consisted of 75 unrelated patients of Belgian origin ascertained at the Erasme Hospital in Brussels, and 75 age, gender and ethnicity matched control subjects recruited through announcements in the hospital. All control individuals were interviewed to exclude psychiatric conditions. Patients fulfilled the Research Diagnostic Criteria¹⁶ for BP disorder.

B. PCR Amplification

Genomic DNA and cDNA were amplified using six overlapping primer sets spanning the CAP2 cDNA sequence (GenBank acc. no L40377).¹⁵ Approximately 50 ng of genomic DNA or 1 ng of cDNA and 10 pg of each primer were used in a standard PCR reaction. Amplification conditions were as follows: initial denaturation step at 94° C. for 4 min, followed by 35 cycles at 94° C. for 1 min, 55° C. for 1 min, 72° C. for 2 min, and a final extension time at 72° C. for 10 min.

C. Southern Blot Analysis

Genomic DNA from 3 affected and 2 non-affected members of family MAD31 was digested with Hind III and Bam HI separately and run on a 1% agarose gel. Southern blotting was performed according to the standard protocol¹⁷.

50 ng of CAP2 cDNA was labeled with (α-³²P) dCTP by random-primed labeling (Gibco-BRL). Hybridization was carried out overnight in Church buffer at 65° C. Subsequently, membranes were washed one time in 1×SSC, 0.1% SDS, one time in 0.5×SSC, 0.1% SDS and two times in 0.1×SSC, 0.1% SDS at 65° C. followed by exposure to Kodak X-ray film at −70° C. for 72 h.

D. SSCP and DHPLC Analysis

PCR amplified DNA was analyzed by SSCP using the DNA Analysis System with precast ready-to-use gels and Hydrolink 5% glycerol gels (Pharmacia Biotech). Denaturing high-performance liquid chromatography (DHPLC) was performed on automated instrumentation purchased from Transgenomic (Santa Clara, Calif., USA). Crude PCR products, were loaded on a DNASep column and eluted from the column using an acetonitrile gradient in a 0.1 M triethylamine acetate buffer (TEAA), pH 7, at a constant flow rate of 0.9 ml/min. The gradient was created by mixing eluents A and B. Eluent A was 0.1 M TEAA, 0.1 M Na₄EDTA. Eluent B was 25% acetonitrile in 0.1 M TEAA. The gradient and temperature required for successful resolution of heteroduplex molecules were predicted by Wavemaker version 3.4.4.

E. DNA Sequencing

Sequencing was performed on plasmid DNA or gel purified PCR templates using a Perkin-Elmer ABI 377 automated sequencer and the Big Dye terminator cycle sequencing kit (Applied Biosystems, PE), according to the manufacturer's protocol. PCR fragments were first visualized on an agarose gel and then gel purified, using Ultrafree-DA filter devices (Millipore).

F. Pyrosequencing

Biotinylated PCR products were immobilized onto streptavidin-coated paramagnetic beads (Dynal AS, Oslo, Norway). ssDNA was obtained by incubating the immobilized PCR product in 50 μl 0.5 M NaOH for 5 min. followed by 2 sequential washes in 100 μl 10 mM Tris-Acetate pH 7.6. Primer Exon7-1025 (5′-GTG CCT CTG TCC AAG GTT GC-3′) was used as pyrosequencing primer for the detection of the SNP c.942 in exon7. Primer annealing was performed by incubation at 72° C. for 2 min and then at room temperature for 5 min. Pyrosequencing was performed on the PSQ96 pyrosequencer (Pyrosequencing AB, Uppsala, Sweden).

G. Statistical Analysis

Total allele and genotype distributions of BP cases and controls were compared and Hardy-Weinberg equilibrium was tested using Genepop.¹⁸ Allele and genotype specific comparisons were done using a chi-square analysis or where appropriate, a Fisher exact test. The Dismult program ¹⁹ was used for multipoint association analysis combining the data of all SNPs genotyped. Linkage disequilibrium (LD) was calculated using Linkdos of Genepop. To estimate haplotype frequencies in patients and controls, the Arlequin algorithm based on the maximum likelihood method was applied.²⁰

H. Genomic Structure of CAP2 Gene

We determined the genomic structure of the CAP2 gene. First, the CAP2 gene was analyzed for genomic rearrangements by hybridizing a PCR-derived CAP2 cDNA fragment against two different Southern blots containing Hind III- and Bam HI-digested genomic DNA from the 5 selected members of family MAD31 (3 affected and 2 unaffected). Based on the observed hybridized bands, a minimal genomic size of 25 kb was estimated. No difference between the hybridization patterns of affected and non-affected individuals were observed.

Using cDNA primers, the position and size of introns were obtained from PCR on genomic DNA. After sequencing, the exact exon-intron boundaries were determined by comparison of cDNA and genomic sequences (Table 1). Intronic primers were designed from these genomic sequences (Table 4).

TABLE 4
Intronic CAP2 primers

| | |
|---|---|
| PCR primers | |
| for Exon 3 | |
| Forward | 5′ ACTTTCAAT TTCTTTGTCATC 3′ |
| | (SEQ ID NO 3) |
| Reverse | 5′ TACAAAGCAGGAGATATTCACC 3′ |
| | (SEQ ID NO 4) |
| PCR primers | |
| for Intron 4 | |
| Forward | 5′ GAAGCATATAAATGACTGGGTG 3′ |
| | (SEQ ID NO 5) |
| Reverse | 5′ GATAAGAAATGACAGAGTTGC 3′ |
| | (SEQ ID NO 6) |
| PCR primers | |
| for Exon 5 | |
| Forward | 5′ CCAAGAGAATATTTCCTG 3′ |
| | (SEQ ID NO 7) |
| Reverse | 5′ AGTCGATCCCCTGACAAAGC 3′ |
| | (SEQ ID NO 8) |
| PCR primers | |
| for Exon 7 | |
| Forward | 5′ AGCTGGAGGAGAGTTATGACTT 3′ |
| | (SEQ ID NO 9) |
| Reverse | 5′ GCAAGATAGGTAGAAGGAAAGG 3′ |
| | (SEQ ID NO 10) |

This analysis showed that CAP2 contains 1 non-coding and 6 coding exons with sizes ranging from 73 bp (exon 1) to 405 bp (exon 7) (FIG. 1 and table 1). The sizes of introns 2 to 6 were determined by PCR and ranged from 1.3 Kb (intron 2) to 1.8 Kb (intron 3) (FIG. 1). While these experiments were in progress, the complete sequence of the BAC 793J2 containing the CAP2 gene became available (Genbank Acc. no AC009802). Exon-intron boundaries sequences, intron and exon sizes were confirmed and the size of intron 1 was determined at 8.1 kb. In total, the CAP2 gene spans a genomic region of 17.1 kb.

To establish the orientation of the CAP2 gene, a CAP2-CAG fragmented YAC²¹ was analyzed for the presence of STS markers centromeric and telomeric to the gene including L40377 (CAP2 exon 7). PCR analysis showed positive hits with markers centromeric to CAP2 and the absence of amplification with markers telomeric to the gene indicating that the transcription orientation of CAP2 is from centromere to telomere.

TABLE 1
Intron-Exon Boundaries in CAP2

| | | | |
|---|---|---|---|
| Exon N^{o} | Size (bp) | Splice acceptor | Splice donor |

| | | | |
|---|---|---|---|
| 1 | 73 | | GCAGCAGGAG/gtgggggcct |
| 2 | 178 | tttgatgcag/ACCTTCTCTG | GATGTCCCAG/gtatgtgtgc |
| 3 | 138 | tttgatgcag/ACCTTCTCTC | TTCCTTCCAG/taagtagtat |
| 4 | 118 | gtgtttgcag/GACTTTAAAGA | AAGACTGAAG/gtgagacagt |
| 5 | 143 | ttctttatag/GTAAGATTTC | AACCAACGAG/gtagggaaag |
| 6 | 153 | tttccgttag/GAAAAAAAGA | CCTCGCCGTG/gtaagctcca |
| 7 | 405 | cttatcctag/GTGGAAAAAG | TTCTCCGTAA |

Mutation Detection and Analysis

Intronic primers were designed in order to PCR amplify all exons from DNA of 24 BP patients. PCR products were screened for mutations using SSCP analysis. Two non-synonymous SNPs were identified at c.203G>A (Arg68G1n) and c.1076G>A (Arg359His). One synonymous SNP coding for Leu was identified c.477A>G (codon 159). In addition, 1 SNP was detected in intron 4, IVS4+98A>G. These results were confirmed by DHPLC analysis and resulted in the identification of 2 additional SNPs in exon 7, at c.910A>G (Thr304Ala) and one in c.942C>T (Ala314Ala) coding for Ala (Table 2).

TABLE 2
CAP2 polymorphisms

| | | | Protein | Amino |
|---|---|---|---|---|
| Location of | Nucleotide | Restriction site | position | acid |
| Polymorphism | position cDNA | change | codon | change |
| Exon 3 | c.203G > A | | 68 | Arg to Gln |
| Intron 4 | IVS4 + 98A > G | Gain of Rsa I | | |
| Exon 5 | c.477A > G | Loss of Mae I | 159 | Leu to |
| | | | | Leu |
| Exon 7 | c.910A > G | Gain of Pvu II | 304 | Thr to Ala |
| Exon 7 | c.942C > T | | 314 | Ala to Ala |
| Exon 7 | c.1076G > A | Loss of Hha I | 359 | Arg to His |

PCR-RFLP analysis in 75 unrelated bipolar patients and 75 matched controls was performed for 3 of these variants: A Rsa I-RFLP assay was applied for the SNP IVS4+98A>G. A Pvu II-RFLP analysis was applied for SNP c.910A>G. A Hha I-RFLP analysis was applied for the SNP c.1076G>A. SNPs c.477A>G and c.203G>A were analyzed by direct sequencing of PCR fragments generated from genomic DNA. Pyrosequencing was used to analyze the SNP c.942C>T.

There was no significant difference between BP patients and controls in allele frequencies or genotype distribution in 5 of these SNPs. However, there is a slight departure of Hardy-Weinberg equilibrium for SNPs c.203G>A (p=0.05) and c.477A>G (p=0.03), both in BP, which comes from an excess of heterozygotes (p=0.03 for ex3; p=0.02 for ex5). In addition there is a slight excess of heterozygotes for SNP c.203G>A in the controls (p=0.04).

The T allele of SNP c.942C>T had a significantly higher frequency in BP cases (6%) than in controls (1%) (χ²=4.83;p=0.03). When comparing genotypes 9/73 (12%) had one T allele compared with 2/75 (3%) controls (χ²=5.02; p=0.03). Interestingly, when data was analyzed after stratification for gender, a significant difference was observed. In males the T allele had a frequency of 8% in BP patients while it was not observed in controls (Fisher exact test, p=0.03). In females no difference in allele or genotype distribution was observed between cases and controls.

To confirm these results the unrelated patients and matched control groups were extended and final genotyping association analysis performed in 113 BP patients and 163 age, sex and ethnicity matched controls. Table 3 shows the allele and genotype frequencies for these polymorphisms in patient and control populations.

TABLE 3
Genotype and allele frequencies for SNPs in the CAP2 gene

| | Genotypes | | | | | | | Alleles | | |
|---|---|---|---|---|---|---|---|---|---|---|
| | Cases | | | Controls | | | | BP | Controls | |
| Belgian BP | AA | AG | GG | AA | AG | GG | | A | A | |
| SNP | % | % | % | % | % | % | p value | % | % | p value |
| c.203G > A | 2 | 48 | 50 | 2 | 49 | 49 | 0.90 | 26 | 27 | 0.81 |
| IVS4 + 98A > G | 71 | 29 | 0 | 68 | 32 | 0 | 0.63 | 86 | 84 | 0.66 |
| c.477A > G | 5 | 47 | 48 | 8 | 43 | 49 | 0.76 | 28 | 30 | 0.77 |
| c.910A > G | 80 | 19 | 1 | 79 | 20 | 1 | 1.00 | 89 | 89 | 1 |
| c.942C > T | 0 | 10 | 90 | 0 | 3 | 97 | 0.02 | 5 | 1 | 0.02 |
| c.1076G > A | 21 | 43 | 36 | 17 | 50 | 33 | 0.93 | 43 | 42 | 0.93 |

### REFERENCE LIST

The following publications are herein incorprated by reference.
1. Akiskal, H. S. et al. Psychopathology, temperament, and past course in primary major depressions. 1. Review of evidence for a bipolar spectrum. Psychopathology 22, 268-277 (1989).
2. Tsuang M T & Faraone S V. The Genetics of Mood Disorders. The Johns Hopkins University Press, Baltimore (1990).
3. Spence, M. A. et al. Bipolar disorder: evidence for a major locus [see comments]. Am. J. Med Genet. 60, 370-376 (1995).
4. Conneally P M. Genetics and Psychiatric Disorders. (1998).
5. McInnis, M. G. et al. Anticipation in bipolar affective disorder. Am. J. Hum. Genet. 53, 385-390 (1993).
6. Nylander, P. O., Engstrom, C., Chotai, J., Wahlstrom, J. & Adolfsson, R. Anticipation in Swedish families with bipolar affective disorder. J. Med. Genet. 31, 686-689 (1994).
7. Li, R. & el-Mallakh, R. S. Triplet repeat gene sequences in neuropsychiatric diseases. Harv. Rev. Psychiatry 5, 66-74 (1997).
8. Van Broeckhoven, C. & Verheyen, G. Chromosome 18 workshop. Psychiatr. Genet. 8, 97-108 (1998).
9. Van Broeckhoven, C. & Verheyen, G. Report of the chromosome 18 workshop. Am. J. Med. Genet. 88, 263-270 (1999).
10. De bruyn, A., Souery, D., Mendelbaum, K., Mendlewicz, J. & Van Broeckhoven, C. Linkage analysis of families with bipolar illness and chromosome 18 markers. Biol. Psychiatry 39, 679-688 (1996).
11. Verheyen G R et al. Genetic refinement and physical mapping of a chromosome 18q candidate region for bipolar disorder. Eur. J. Hum. Genet. 7, 427-434 (1999).
12. Huber, R. & Carrell, R. W. Implications of the three-dimensional structure of alpha 1-antitrypsin for structure and function of serpins. Biochemistry 28, 8951-8966 (1989).
13. Remold-O'Donnell, E. The ovalbumin family of serpin proteins. FEBS Lett. 315, 105-108 (1993).
14. Bartuski, A. J., Kamachi, Y., Schick, C., Overhauser, J. & Silverman, G. A. Cytoplasmic antiproteinase 2 (PI8) and bomapin (PI10) map to the serpin cluster at 18q21.3. Genomics 43, 321-328 (1997).
15. Sprecher, C. A. et al. Molecular cloning, expression, and partial characterization of two novel members of the ovalbumin family of serine proteinase inhibitors. J. Biol. Chem. 270, 29854-29861 (1995).
16. Endicott J & Spitzer R L. Schedule for Schizophrenia and Affective Disorders-Lifetime Version. New York:N.Y. state Psychiatric Institute., N.Y. (1978).
17. Sambrook J, Fritsch E & and Maniatis T. Molecular Cloning: A Laboratory Manual. Cold Spring Harbor, N.Y. (1989).
18. Raymond, M. & Rousset, F. GENEPOP (version 1.2): a population genetics software for exact tests and ecumenicism. Journal of Heredity 86, 248-249 (1995).
19. Terwilliger, J. D. A powerful likelihood method for the analysis of linkage disequilibrium between trait loci and one or more polymorphic marker loci. Am. J. Hum. Genet. 56, 777-787 (1995).
20. Schneider S, Roessli D & Excoffier L. Arlequin ver. 2.000: A software for population genetics data analysis. (2000).
21. Goossens, D. et al. No evidence for the involvement of CAG/CTG repeats from within 18q21.33-q23 in bipolar disorder. Eur. J. Hum. Genet. 8, 385-388 (2000).

1. A method of diagnosing BP or susceptibility to BP in an individual which method comprises determining, in a sample from the individual, the single nucleotide polymorphism in the CAP2 gene of the individual, and determining the status of the individual by reference to polymorphism in the CAP2 gene.

2. A method according to claim 1 wherein the single nucleotide polymorphism of the individual is in linkage disequilibrium with the polymorphism in the CAP2 gene.

3. A method according to claim 1 wherein the single nucleotide polymorphism equals SNP c.942G>T.

4. A method according to claim 1 wherein the single nucleotide polymorphism in the CAP2 gene of the individual is determined by a method selected from amplification refractory mutation system and restriction fragment length polymorphism such as Southern blotting techniques, single-strand conformational polymorphism analysis, chemical cleavage of mismatches and denaturing high-performance liquid chromatography.

5. A method according to claim 1 wherein the single nucleotide polymorphism in the CAP2 gene of the individual is determined using a pair of PCR primers that amplify a fragment of the CAP2 gene containing the single nucleotide polymorphism.

6. A method according to claim 5 wherein the single nucleotide polymorphism consists of SNP c.942G>T.

7. A method according to claim 5 wherein the pair of PCR primers that amplify a fragment of the CAP2 gene consist of a forward and reverse primer comprising the sequences of SEQ ID No 9 and SEQ ID No 10.

8. A pair of PCR primers capable of amplifying a fragment of the CAP2 gene containing the single nucleotide polymorphism.

9. A pair of PCR primers consisting of a forward and reverse primer comprising the sequences of SEQ ID No 9 and SEQ ID No 10.

10. A diagnostic kit comprising the pair of PCR primers according to claim 8.

11. A method according to any one of claim 1 wherein the single nucleotide polymorphism in the CAP2 gene of the individual is determined by means of an allele-specific oligonucleotide probe.

12. An allele-specific oligonucleotide probe capable of detecting the single nucleotide polymorphism SNP c.942G>T in the CAP2 gene of an individual.

13. A diagnostic kit comprising the allele-specific oligonucleotide probe according to claim 12.
